(19) United States
(12) Patent Application Publication
(10) Pub. No.: US 2006/0263431 A1
(43) Pub. Date: Nov. 23, 2006

(54) Opioid Sustained Release Formulation

(75) Inventor: Ann M. Maloney, Dublin, OH (US)

Correspondence Address: MICHAEL P. MORRIS;BOEHRINGER INGELHEIM CORPORATION, 900 RIDGEBURY ROAD, P O BOX 368, RIDGEFIELD, CT, 06877-0368, US

(73) Assignee: Roxane Laboratories, Inc., Columbus, OH (US)

(21) Appl. No.: 11/462,061

(22) Filed: Aug. 3, 2006

Related U.S. Application Data
(63) Continuation of application No. 10/657,011, filed on Sep. 5, 2003, now abandoned.
Application No. 10/657,011 is a continuation of application No. 10/085,597, filed on Feb. 27, 2002, now abandoned.
Application No. 10/085,597 is a continuation of application No. 09/626,584, filed on Jul. 27, 2000, now abandoned.
(60) Provisional application No. 60/146,298, filed on Jul. 29, 1999.

(30) Foreign Application Priority Data
Jul. 27, 2000 (WO) PCT/US00/20413

(51) Int. Cl. A61K 31/485; A61K 9/14

(52) U.S. Cl. 424/486; 424/487; 514/282; 424/488

(57) ABSTRACT

A solid, oral, controlled release dosage form comprising a therapeutically effective amount of an opioid compound, or a salt thereof, a matrix-forming polymer and an ionic exchange resin.

### BACKGROUND OF THE INVENTION

1. Field of Invention

The present invention relates to an improved pharmaceutical drug delivery composition. More particularly, the present invention is directed to a controlled release formulation, capable of providing sustained, prolonged, repeat and/or delayed release, and methods for preparing the same. Such formulations have improved delivery characteristics.

2. Background of the Related Art

It is well known in the art that the maximum time of effectiveness of many pharmaceutical formulations, including conventional opioid formulations, is only a few hours because of biological modification or elimination of the drug from the body. Consequently, doses of such pharmaceutical formulations must be taken at frequent intervals to obtain long term therapeutic levels of active drug component.

Many attempts have been made to design sustained-release pharmaceutical preparations to provide a more constant level of the drug in the blood over a set period of time. Many sustained-release preparations were originally contemplated as “convenience dosage forms,” that is, dosage forms designed to improve QOL (that is, the “quality of life”) of a patient by eliminating the necessity of dosing a patient several times during the day and by proffering the advantage of decreased missed doses which might result from the forgetfulness of a patient. A number of such preparations, however, have subsequently been shown to provide clear therapeutic benefits which cannot be obtained by multiple dosing of their active drug component (especially those drugs which display high water solubility).

Among the many possible therapeutic benefits provided by sustained-release dosage forms are: (1) the allowance of more constant blood levels over time (thus avoiding large spike and trough levels not infrequently seen with rapidly dissolving dosage forms) leading to a more consistent therapeutic effect; (2) delay of the release of drug such that significant absorption of the drug may occur at more desirable sites (e.g., causing the bulk of the absorption to occur in a more desirable pH milieu and thus reducing decomposition of the drug); (3) reduction in concentration dependent gastrointestinal irritation (owing to reduction in the concentration of drug in contact with a particular surface of the gastrointestinal tract); and (4) improvement of drug safety with respect to acute toxicity owing to lower concentrations of drug being released at a particular time as compared to readily available dosage forms of similar dose.

Numerous methods have been described to prepare sustained release formulations of drugs.

One of the most common techniques for delaying release of a drug from a pharmaceutical preparation is to incorporate the drug into a continuous matrix which is resistant to rapid dissolution by aqueous body fluids. The release of the drug in such matrix-based sustained-release preparations is driven by the drug concentration gradient resulting from diffusion of fluid into the dosage form. The matrices may be comprised of either erodable polymers (i.e., polymers that break down in the body) or non-erodable polymers (polymers that are substantially unchanged upon passage through the gastrointestinal tract). While commonly employed, an intrinsic problem with many matrix release preparations is that at the later stage of release the rate of release is disadvantageously diminished as a result of decrease in the concentration gradient across the surface of the tablet, and an increase in the distance of diffusion (a problem which is particularly associated with non-erodable polymers).

In one type of matrix system, sustained release is effectuated by mixing the active drug product with one or more hydrophilic hydrocolloids such that when the hydrocolloids are contacted with gastric fluid at body temperature, a sustained gelatinous mix is formed on the surface of the dosage form. The gelatinous layer reduces the dissolution rate and eventuates in slow release of the drug from the surface of the dosage form. For example, U.S. Pat. Nos. 3,965,256 and 4,235,870 teach slow release pharmaceutical compositions employing hydroxyalkyl cellulose and a higher aliphatic alcohol, while U.S. Pat. No. 4,140,755 to Sheth et al. discloses sustained release tablets utilizing hydroxypropylmethylcellulose having a viscosity of 4000 cps. An advantage of hydroxypropylmethylcellulose (a series of compounds designated as Methocel E, F, J and K, each of which has a different chemical composition with a methoxyl content within the range of 16.5 to 30 weight percent, and a hydroxypropyl content within the range of 4 to 32 weight percent) matrix formulations is that drug release rates are generally independent of processing variables such as compaction pressure, drug particle size and the incorporation of lubricant (See, Feely et al., Int. J Pharmaceutics 41 (1988) 83-90). Admixture of hydroxypropylmethylcellulose with anionic surfactants is reported to improve prolongation of drug release (See, Alli et al., J. Applied Polymer Science 42 (1991) 947 956; U.S. Pat. No. 4,795,327). Drug release kinetics in swellable matrices can be described by a second order equation in which polymer chain relaxation and drug diffusion influence the release behavior (See, Colombo et al., Int. J. Pharmaceutics 88 (1992) p. 99-109). Release kinetics, however, can be changed towards linearity by slowing matrix swelling achieved through adjusting the external matrix surface. Id.

Another common approach to form sustained-release preparations is to microencapsulate the drug in a polymeric composition thus providing a slower dissolution rate. Microcapsules are designed such that the gastric fluids slowly diffuse through the capsule walls, dissolving the active drug. The dissolved drug slowly diffuses or leaches out through the microcapsule wall into the body. U.S. Pat. Nos. 3,155,590, 3,341,416, 3,488,418 and 3,531,418 are representative of early work involving microencapsulation techniques. While microencapsulation is used extensively in sustained-release formulations, microencapsulation of drugs frequently fails to provide a desired sustained-release profile in that the dissolution rate often decreases rapidly over time. Efforts to adjust the rate of dissolution from microcapsules and, thus, control the timing of sustained release, are disclosed, for example, in U.S. Pat. No. 3,492,397 wherein the dissolution rate is said to be controlled by adjusting the wax/ethyl cellulose ratio, U.S. Pat. No. 4,752,470 wherein the controlled release characteristics are varied by altering the ratio of ethyl cellulose to hydroxypropyl cellulose in the coating, and U.S. Pat. No. 4,205,060 wherein it is disclosed that the rate of dissolution of various drugs can be controlled by varying the thickness of the coating applied to those drugs.

It is also known in the art to prepare sustained release formulations of medicaments by applying rupturable, relatively water-insoluble, water permeable films over an insoluble swelling type release matrix (such as a blend of polyvinyl pyrrolidone and carboxyvinyl hydrophilic polymer) which contains the medicament (See, e.g., U.S. Pat. No. 4,252,786 to Weiss). Sustained release formulations containing actives in a coated core material are also known (See, e.g., U.S. Pat. Nos. 4,248,857 and 4,309,405)

Multilayering is also used to prepare solid dosage forms with sustained release profiles. Such technique involves incorporating into the dosage form two or more separate layers of granulation which are designed to release drug at different rates. By compounding each layer differently, the rate of dissolution of the layer may be controlled in a desired manner.

Controlled drug release may also be effectuated by taking advantage of charge-charge interactions, such as reacting basic drugs with polymers having acidic moieties (See, e.g. U.S. Pat. No. 3,608,063). For example, extended action has been obtained by loading drugs onto ion-exchange resins (See, Remington's Pharmaceutical Sciences, 15^{th} Ed. 1975). Such extended action is presumed to result from the slow rate of the displacement reaction when drug-resin complex contacts gastrointestinal fluids and ionic constituents are displaced from the resin, essentially by other ions. Sorption of the drug to the resin is believed to be primarily due to ionic electrostatic interactions (See, Jenquin et al., Int. J. of Pharmaceutics 101 (1994) 23-34). Thus for example, amine containing drugs (such as codeine (See, e.g. Amsel et al., Pharm. Tech. 8 (1984) 28) and propanolol (Burke et al, Drug DeveL Indust. Pharmacy 12 (1986) 713-732)) may be bound to strong cationic exchange resins yielding restricted elution of the drug from the resinates (See, Sanghvai et al., Indian Drugs 26 (1988) 27-32). Uncoated ion exchange resin-drug complexes which delay release of a drug in the gastrointestinal tract are described in U.S. Pat. Nos. 2,990,332, 3,138,525, 3,499,960, 3,594,470, Belgian Pat. No. 729,827, German Pat. No. 2,246,037 and Brodkins et al., Journal of Pharmaceutical Science, Vol. 60, pages 1523-1527 (1971).

The problem with early ion exchange resin-drug compositions was that the drug complexes were often too rapidly released in the gastrointestinal tract. Attempts to reduce the release rate by use of diffusion barrier coatings were frequently found to be ineffective as the coatings were often found to peel rapidly from the complex as the complex swelled upon exposure to biological fluids. Numerous proposals have been proffered in the context of barrier-coated ion exchange resin-drug formulations to decrease the release rate including the incorporation of solvating agents, such as polyethylene glycol, higher aliphatic alcohols, and matrix-forming cellulose ethers in formulation of the resin-drug complex (See, e.g., U.S. Pat. No. 4,221,778, U.S. Pat. No. 4,861,598 and Feely et al., Int. J. Pharmaceutics 44 (1988) 131-139 and Pharmaceutical Research 6 (1989) 274-278, respectively).

There is a growing recognition in the medical community that a large number of patients suffer from the undertreatment of pain. Among the reasons frequently cited as causative of undertreatment are: (1) the failure to prescribe enough drug at the right dosage interval to reach a steady-state threshold commensurate with the pain relief needed; (2) failure of patients to comply with a given dosage regimen; and (3) the reluctance of many physicians to prescribe analgesics categorized as controlled drugs based on often unfounded concerns of future addiction and fear of regulatory review of the physician's prescribing habits. For example, it has been reported that with respect to cancer pain, a large percentage of cancer patients suffer debilitating pain despite treatment with analgesics (Cleeland et al., N. Eng. J. Med. 330 (1994) 592-596).

Opioid analgesics comprise the major class of drugs used in the management of moderate to severe pain. Until recently most opioid analgesics were available only in rapid dissolution forms. Because opioid drugs typically are metabolized and/or excreted relatively rapidly, dosing of rapid dissolution opioid preparations is typically frequent so that steady state blood levels may be maintained. Due to rapid dissolution and absorption which results in a relatively large peak to trough differential with regard to active drug concentrations, pain relief from rapid dissolution opioids is frequently found to be quite variable.

Several manufacturers presently market sustained-release opioid analgesic formulations to overcome one or more of the problems associated with the administration of rapid dissolution opioids. Sustained-release opioid formulations promise relief from pain with, in theory, minimal addiction liability owing to a substantially lower Cₘₐₓ without compromise of analgesic efficacy. The approach taken by many manufacturers has been to develop sustained-release opioid formulations which provide zero order pharmacokinetics (thereby providing very slow opioid absorption and a generally flat serum concentration curve over time) to mimic a steady-state level. However, it has been reported that greater analgesic efficacy is achieved by formulations designed to provide more rapid initial opioid release within two to four hours, and which follow first order pharmacokinetics (See, e.g., U.S. Pat. No. 5,478,577). Numerous sustained-release opioid analgesic formulations have been proposed, employing, for example, granulation and coating of the opioid drug (e.g., with a water insoluble cellulose), to control the release of the drug (See, e.g., U.S. Pat. Nos. 5,478,577, 5,580,578, 5,639,476, and 5,672,360), standard release matrices (See, e.g., U.S. Pat. No. 5,226,331), drug loading onto a resin utilizing wet granulation (See, e.g., U.S. Pat. Nos. 4,990,131 and 5,508,042) and hydrophilic matrices in conjunction with one or more aliphatic alcohols (See, e.g., U.S. Pat. Nos. 4,844,909, 4,990,341, 5,508,042, and 5,549,912).

While presently available sustained-release opioid analgesic formulations have improved therapeutic efficacy (i.e., dosing is less frequent and hence dosing compliance by patients is believed to be achieved over rapid dissolution-type dosage forms incorporating the same opioid analgesic) in practice, consistent amelioration of pain between administration of doses is often less than adequate. Further, manufacture of presently available sustained-release opioid analgesic formulations is complex, requiring specialized granulation and coating equipment, cumbersome techniques, and expensive excipients.

There is therefore a need for an improved sustained-release formulation for the release of opioid compounds, and opioid analgesics in particular.

### BRIEF SUMMARY OF THE INVENTION

The present invention provides an improved solid, oral dosage formulation for the in vivo sustained-release of opioid compounds, and salts thereof, and in particular for the sustained-release of opioid analgesics. The formulation comprises a simple mixture of a hydrophilic matrix-forming agent, ionic exchange resin, and one or more opioid compound(s). Such formulation may be prepared without the need for wet granulation of the mixture, drug loading of the resin, or the application of coating materials over the active component. However, wet granulation may be employed. Significantly improved formulations employ ionic exchange resins which are processed such that the particle size distribution of the resin is less than or equal to about 325 mesh, U.S. Standard mesh size, and the mean particle size of the resin particles is less than about 50 μm.

In particular, the present invention provides an improved formulation for the sustained release of oxycodone. An oxycodone formulation of the present invention comprises a therapeutically effective amount of oxycodone, or salt thereof, in a matrix wherein the dissolution rate in vitro of the dosage form, when measured by the USP Basket Method at 100 rpm in 900 mL aqueous buffer (pH 1.2 for the first hour and 7.5 for hours 2 through 12) at 37° C. is between about 5 and 25% (by weight) oxycodone released over the first hour, between about 16 and 36% (by weight) oxycodone released after the second hour, between about 40 and 60% (by weight) oxycodone released after six hours, and between about 60 and 80% (by weight) oxycodone released after twelve hours. The release rate is independent of pH between about 1.2 and 7.5. Additionally, the peak plasma level of oxycodone obtained in vivo occurs between five and six hours after administration of the dosage form.

It has surprisingly been found that formulations having from about 5 to about 100 mg oxycodone may be manufactured to have such release rates when the formulation comprises between about 30 and 65% matrix-forming polymer, more preferably between 50-60% matrix-forming polymer, and between about 1 and 20% ion exchange resin. Significantly improved formulations containing approximately 10 mg-30 mg of oxycodone hydrochloride contain between about 50 to about 60% matrix-forming polymer and between about 5 and about 15% ion exchange resin.

### DETAILED DESCRIPTION OF THE INVENTION

The present invention overcomes many of the prior art problems associated with sustained-release opioid formulations. After considerable experimentation, with numerous conventional sustained-release modalities and techniques (and combinations thereof), the present inventor has discovered a unique sustained-release formulation and process for opioid compounds, and in particular opioid analgesics, which does not require polymeric coatings to be applied to the active, does not require wet granulation procedures in the preparation of the formulation (although wet granulation can be used if desired), and does not require drug loading onto exchange resins, and yet which provides an advantageous release profile of the active.

In a first aspect of the invention, there is disclosed a solid, oral, controlled release dosage form comprising a therapeutically effective amount of opioid compound, or a salt thereof, between about 30 and 65% of a matrix-forming polymer, more preferably between about 50-60% matrix-forming polymer, and between 5 and 15% of a ionic exchange resin. Preferably the opioid compound included in the formulation is an opioid analgesic. It has been surprisingly found that a simple mixture of the matrix-forming agent with the opioid compound and ion-exchange resin, in the proportions disclosed, results in a formulation with improved opioid release kinetics without the need for, or recourse to, expensive coating procedures or wet granulation techniques. Coating and wet granulation may be used in conjunction with the present invention in order to obtain desired tablet configurations, but such procedures and techniques are optional. Such discovery is taught away from by presently available opioid analgesic sustained-release preparations, and goes against conventional thought with respect to highly water soluble drugs (such as the opioid analgesics) which points toward the desirability of drug loading onto the resin, of coating drug-resin complexes, and which suggests that uncoated complexes provide only a relatively short delay of drug release (See, e.g., U.S. Pat. No. 4,996,047 to Kelleher et al.). The present invention also provides a pharmaceutical preparation with a different pharmacokinetic profile. Peak plasma levels of, for example, oxycodone, five to six hours after administration presents a unique profile for an analgesic.

By the term “opioid,” it is meant a substance, whether agonist, antagonist, or mixed agonist-antagonist, which reacts with one or more receptor sites bound by endogenous opioid peptides such as the enkephalins, endorphins and the dynorphins. By the term “opioid analgesic” it is meant a diverse group of drugs, of natural, synthetic, or semi-synthetic origin, that displays opium or morphine-like properties. Opioid analgesics include, without limitation, morphine, heroin, hydromorphone, oxymorphone, buprenorphine, levorphanol, butorphanol, codeine, dihydrocodeine, hydrocodone, oxycodone, meperidine, methadone, nalbulphine, opium, pentazocine, propoxyphene, as well as less widely employed compounds such as alfentanil, allylprodine, alphaprodine, anileridine, benzylmorphine, bezitramide, clonitazene, cyclazocine, desomorphine, dextromoramide, dezocine, diampromide, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, remifentanil, hydroxypethidine, isomethadone, ketobemidone, levallorphan, levophenacylmorphan, lofentanil, meptazinol, metazocine, metopon, myrophine, narceine, nicomorphine, norpipanone, papvretum, phenadoxone, phenomorphan, phenazocine, phenoperidine, piminodine, propiram, sufentanil, tramadol, tilidine, and salts and mixtures thereof.

Matrix-forming polymers useful in the present invention may comprise any polymer not readily degradable by the body. Typical matrix-forming polymers useful in the present invention, include, without limitation, hydroxypropylmethyl cellulose (in particular having a molecular weight range of 50,000 to 1,250,000 daltons), ethylcellulose, methylcellulose, hydroxypropyl cellulose, hydroxyethyl cellulose, carboxymethyl cellulose calcium, sodium carboxymethylcellulose, hydroxypropylmethyl cellulose phthalate, cellulose acetate phthalate, camauba wax and stearyl alcohol, carbomer, cetostearyl alcohol, cetyl alcohol, cetyl esters wax, guar gum, hydrogenated castor oil, magnesium aluminum silicate, maltodextrin, polyvinyl alcohol, polyvinyl chloride, polyethylene glycol, polyethylene glycol alginate, polymethacrylates, polyesters, polysaccharides, poloxamer, povidone, stearyl alcohol, glyceryl stearate, gelatin, acacia, dextran, alginic acid and sodium alginate, tragacanth, xanthan gum and zein. A preferred matrix-forming polymer is alkylcellulose-based, more particularly hydroxyalkylcellulose-based. Alkylcellulose matrix-forming polymers were found unexpectedly to improve the release profile of opioids when used in conjunction with numerous types of ionic exchange resins. The most efficacious matrix-forming polymers were found to be hydrophilic in nature.

Among the ionic exchange resins useful in the present invention, without limitation, are styrene-divinylbenzene copolymers (e.g. IRP-69, IR-120, IRA-400 and IRP-67—Rohm & Haas), copolymers of methacrylic acid and divinylbenzene (e.g. IRP-64 and IRP-88—Rohm & Haas), phenolic polyamines (e.g., IRP-58—Rohm & Haas), and styrene-divinylbenzene (e.g., colestyramine resin U.S.P.). The drug and resin should be oppositely charged such that the drug will bind to the resin when solubilized in the matrix formed by the matrix-former. As most opioid compounds are basic in nature, it is preferred that the ionic exchange resin be cationic in nature, and most preferably be strongly acidic in nature.

It has been surprisingly found that micronization of the ionic resin particles, such that about 90% or more of the particles are less than about 325 mesh, U.S. Standard mesh size, or such that the particles have an mean particle size of less than about 50 μm, significantly improves the sustained release profile of a wide array of opioid compounds incorporated into a polymeric matrix, in particular a hydrophilic matrix. A further aspect of the present invention therefore comprises a novel solid, oral, controlled release dosage form comprising a therapeutically effective amount of an opioid compound, or a salt thereof, between about 30 and 65% of a matrix-forming polymer and between 5 and 15% ionic exchange resin having a mean particle size of less than about 50 μm and a particle size distribution such that not less than 90% of the particles pass through a 325 mesh sieve, US. Standard Sieve Size. In particular, the present inventor has found that strongly acidic cationic exchange resins, such as IRP-69 (Rohm & Hass), having a particle size of less than about 325 mesh (U.S. Standard mesh size) and/or a mean particle size of less than about 50 μm, more preferably less than about 44 μm, are particularly useful in formulating improved slow-release oxycodone preparations, particularly when an alkylcellulose matrix-former is utilized.

The formulations of the present invention may include diluents, lubricants, glidants and additives, as known to those of ordinary skill in the art to improve compaction, augment swallowability, decrease gastrointestinal irritation, and generally to improve the pharmaceutical elegance of the final product. Among the diluents which may find application in the present formulations are, without limitation, lactose, microcrystalline cellulose, starch and pregelatinized starch, sucrose, compressible sugar and confectioner's sugar, polyethylene glycol, powdered cellulose, calcium carbonate, calcium sulfate, croscarmellose sodium, crospovidone, dextrates, dextrin, dextrose, fructose, glyceryl palmitostearate, kaolin, magnesium aluminum silicate, magnesium carbonate, magnesium oxide, maltodextrin, mannitol, dibasic calcium phosphate, tribasic calcium phosphate, sodium strach glycolate, sorbitol, and hydrogenated vegetable oil (type 1). Among the lubricants which may find application in the present formulations are, without limitation, stearic acid, calcium stearate, glyceryl monostearate, glyceryl palmitostearate, hydrogenated castor oil, hydrogenated vegetable oil (type 1), magnesium stearate, sodium stearyl fumarate, talc and zinc stearate. Suitable glidants, which may find application in the present formulations, are, without limitation, colloidal silicon dioxide, magnesium trisilicate, starch, talc, and tribasic calcium phosphate. Among the many additives that may find application in the present formulations are, without limitation, colorants, flavorants, sweetners, granulating agents, and coating agents such as cellulose acetate phthalate. A formulation of the present invention may comprise from about 0.1-500 mg opioid compound, a matrix-forming polymer from about 10-95% w/w, an ion exchange resin from about 0.1-50% w/w, a diluent from about 0-100% w/w, a glidant from about 0-5% w/w and a lubricant from about 0-20% w/w.

An advantage of the present formulations is that preparation of the formulations typically requires only industry standard equipment.

Another aspect of the present invention is a process for the preparation of a solid, controlled release, oral dosage form comprising the step of incorporating an analgesically effective amount of an opioid analgesic, or salt thereof, in a bulk mixture comprising about 30 to about 65% of a matrix-forming polymer and about 5 to about 15% of a ionic exchange resin, thereby forming an admixture. Further disclosed is a process for the preparation of a solid, controlled release, oral dosage form comprising the step of incorporating an analgesically effective amount of oxycodone, or a salt thereof, in a bulk mixture comprising about 30 to about 65% of a matrix-forming polymer and about 5 to about 15% of an ionic exchange resin, wherein the dissolution rate in vitro, when measured by the USP Basket Method at 100 rpm in 900 mL aqueous buffer (pH 1.2 for the first hour and 7.5 for hours 2 through 12) at 37° C. is between about 5 and 25% (by weight) oxycodone released over the first hour, between about 16 and 36% (by weight) oxycodone released after the second hour, between about 40 and 60% (by weight) oxycodone released after six hours, and between about 60 and 80% (by weight) oxycodone released after twelve hours. The release rate is independent of pH between about 1.2 and 7.5. Additionally, the peak plasma level of oxycodone obtained in vivo occurs between five and six hours after administration of the dosage form.

Yet another aspect of the present invention relates to methods for reducing the range in daily dosages required to control pain in a human using the formulations described. One method comprises administering an oral controlled release dosage form comprising a therapeutically effective amount of an opioid compound, or salt thereof, between 30 and 65% of a matrix-forming polymer and between 5 and 15% ionic exchange resin. Another method comprises administering a solid, oral, controlled release dosage form comprising a therapeutically effective amount of oxycodone, or a salt thereof, a matrix-forming polymer and a ionic exchange resin comprising a copolymerization of divinylbenzene.

### DETAILED DESCRIPTION OF THE PREFERRED EMBODIMENTS

Certain preferred embodiments of the present invention have been elucidated after numerous experiments. The preferred matrix-forming polymer of the present formulations is an alkylcellulose, more preferably a C₁-C₆ hydroxyalkylcellulose. In a preferred dosage form the hydroxyalkylcellulose is selected from the group consisting of: hydroxypropylcellulose, hydroxypropylmethyl cellulose and hydroxyethylcellulose. While the ionic exchange resin of the present invention may be phenolic-based polyamine condensates or styrene-divinylbenzene co-polymers, it is preferred that the ionic exchange resin comprise a cationic exchange resin, in particular one which is sulfonated, to maximize charge-charge interactions between the resin and the opioids. Cationic exchange resins particularly useful in the present invention may comprise divinylbenzene co-polymers, such as a copolymer of divinylbenzene and styrene, or co-polymer of divinylbenzene and methacrylic acid, and the like. It is preferred that the ionic exchange resin comprise between 5 and 15% of the final dosage form, more preferably between about 7 and 10%. Preferably the final dosage form contains between about 30-65% matrix-forming polymer, more preferably between about 50-60%. The matrix-forming polymer, the opioid compound and ionic exchange resin are preferably admixed with one another in dry form, thus decreasing the time and expense involved in the formulation of a final dosage form. However, coating procedures and wet granulation techniques may optionally be employed. Preferably an oral dosage form is formed by, or in conjunction with, compression and shaping of the admixture. It is preferred, due to the advantageous drug release profile produced thereby, that the ionic exchange resin have a mean particle size of less than about 50 μm and a particle size distribution such that not less than 90% of the particles pass through a 325 mesh sieve, U.S. Standard sieve size. Preferred opioid compounds useful in the present invention are selected, without limitation, from the group consisting of: butorphanol, fentanyl, codeine, dihydrocodeine, hydrocodone bitartrate, hydromorphone, meperidine, methadone, morphine, oxycodone hydrochloride, oxymorphone, pentazocine, propoxyphene hydrochloride and propoxyphene napsylate.

The present inventor has in particular discovered that fine particle size resin, having a particle size such that more than about 90% of the resin particles passes through a 325 mesh screen, U.S. Standard mesh size, significantly improves the sustained release profile of the present formulations as compared to the regular particle size resins (e.g. Amberlite IRP-69M vs. Amberlite IRP-69). For example, biostudies of formulations using fine particle size resin suggest sustained-release formulations of the present invention may provide absorption equivalent to that obtained with oral oxycodone solutions with lower Cₘₐₓ.

Employment of the disclosed formulations with respect to the opioid oxycodone (dihydrohydroxycodeinone) hydrochloride has been found to be particularly advantageous. Oxycodone is a semisynthetic narcotic analgesic agent with actions, uses, and side effects similar to those of hydromorphone and morphine. Typically formulated in conventional tablet form, this highly water soluble compound typically has a half-time of absorption of about 0.4 hours, a half-life of approximately 2 to 3 hours, and a duration of action of approximately 3 to 4 hours.

A particularly useful formulation of oxycodone which has been found to effectively control pain in a wide variety of patients without significant pain breakthrough between doses comprises a solid, oral, controlled release dosage form comprising a therapeutically effective amount of oxycodone, or a salt thereof, a matrix-forming polymer and an ionic exchange resin comprising a divinylbenzene copolymer, wherein the dissolution rate in vitro of the dosage form, when measured by the USP Basket Method at 100 rpm in 900 mL aqueous buffer (pH 1.2 for the first hour and 7.5 for hours 2 through 12) at 37° C. is between about 5 and 25% (by weight) oxycodone released over the first hour, between about 16 and 36% (by weight) oxycodone released after the second hour, between about 40 and 60% (by weight) oxycodone released after six hours, and between about 60 and 80% (by weight) oxycodone released after twelve hours. The release rate is independent of pH between about 1.2 and 7.5. Additionally, the peak plasma level of oxycodone obtained in vivo occurs between five and six hours after administration of the dosage form.

The following examples illustrate various aspects of the present invention. They are not, however, to be construed as limiting the claims in any manner whatsoever.

### EXAMPLE 1

Oxycodone hydrochloride 10 mg sustained-release dosage forms having the formulations given in Table 1 below were prepared as follows: oxycodone hydrochoride, USP, lactose NF (Flast Flo), and Amberlite IRP 69M fine particle size cationic exchange resin were run through a No. 20 mesh screen for delumping and were mixed for 10 minutes. Hydroxypropyl methylcellulose, USP, and Cab-O-Sil (M-5) (a glidant) was passed through a No. 20 mesh screen for delumping and then added to the drug powder blend. Mixing of the admixture was performed for 20 minutes. Stearic Acid NF (powder) (a lubricant) was passed through a No. 40 mesh screen and then added to the mixed batch. The batch was subsequently mixed for 3 minutes, the mixer sides wiped, and any adhering powder incorporated into the batch. The batch was then mixed for an additional 2 minutes and compressed to form tablets.

TABLE 1

| INGREDIENT | FORMULA 1 | | FORMULA 2 | | FORMULA 3 | | FORMULA 4 | |
|---|---|---|---|---|---|---|---|---|
| Oxycodone Hydrochloride | 10 | mg/tablet | 10 | mg/tablet | 10 | mg/tablet | 10 | mg/tablet |
| Lactose, NF (Fast Flo) | 27.8% | w/w | 25.8% | w/w | 31.1% | w/w | 10.8% | w/w |
| Amberlite IRP 69M Fine Particle Size | 5.0% | w/w | 7.0% | w/w | 6.7% | w/w | 20.0% | w/w |
| Methocel K100M (Premium) CR | 55.0% | w/w | 55.0% | w/w | 50.0% | w/w | 50.0% | w/w |
| Cab-O-Sil (M-5) | 0.5% | w/w | 0.5% | w/w | 0.5% | w/w | 0.5% | w/w |
| Stearic Acid, NF (Powder) | 5.0% | w/w | 5.0% | w/w | 5.0% | w/w | 5.0% | w/w |
| Theoretical Tablet Weight | 150 | mg | 150 | mg | 150 | mg | 150 | mg |

The in vitro release rates of formulations 1-4 were assessed by the USP Basket Method described hereinabove. Each of the formulations contained a total of 10 mg of oxycodone hydrochloride. The release rate of oxycodone from each of the preparations is set forth below in Table 2.

TABLE 2

| TIME | FORMULA | FORMULA | FORMULA | FORMULA |
|---|---|---|---|---|
| (HOURS) | 1 (% LA) | 2 (% LA) | 3 (% LA) | 4 (% LA) |
| 0 | 0 | 0 | 0 | 0 |
| 1 | 17.8 | 12.2 | 18.0 | 12.0 |
| 2 | 28.9 | 23.3 | 29.0 | 20.0 |
| 4 | 46.1 | 38.4 | 46.0 | 33.0 |
| 6 | 60.0 | 51.5 | 60.0 | 45.0 |
| 8 | 71.1 | 62.7 | 72.0 | 55.0 |
| 10 | 80.0 | 71.8 | 82.0 | 64.0 |
| 12 | 87.0 | 79.6 | 89.0 | 73.0 |

### EXAMPLE 2

Oxycodone hydrochloride 30 mg sustained-release dosage forms having the formulations given in Table 3 were prepared as follows: Lactose NF (Fast Flo) was passed through a No. 20 mesh screen for delumping and was mixed with the D and C Yellow No. 10 Aluminum Lake 6010 and the FD and C Yellow No. 6 Aluminum Lake 5285 for 10 minutes. The lactose/color mix was then milled. Cab-O-Sil (M-5) (a glidant), oxycodone hydrochloride USP and Amberlite IRP-69M fine particle size were passed through a No. 20 mesh screen for delumping and were then mixed with the lactose/color blend for 10 minutes. Hydroxypropyl methylcellulose USP (Methocel K100M (premium) CR) was passed through a No. 20 mesh screen for delumping then added to the drug powder blend and mixed for 20 minutes. Stearic acid NF (powder) was passed through a No. 40 mesh screen and then added to the batch. The batch was mixed for 3 minutes, then the mixer sides and blades were wiped and adhering powder was incorporated into the batch. The batch was then mixed for an additional 2 minutes and compressed to form tablets.

TABLE 3

| INGREDIENT | FORMULA 5 | | FORMULA 6 | |
|---|---|---|---|---|
| Oxycodone Hydrochloride | 30 | mg/tablet | 30 | mg/tablet |
| Lactose, NF (Fast Flo) | 12.3% | w/w | 14.5% | w/w |
| Amberlite IRP 69M Fine | 10.0% | w/w | 5.0% | w/w |
| Particle Size | | | | |
| Methocel K100M (Premium) | 55.0% | w/w | 55.0% | w/w |
| CR (hydroxylpropyl | | | | |
| methylcellulose, USP) | | | | |
| D and C Yellow No. 10 | 0.4% | w/w | — | |
| Aluminum Lake 6010 | | | | |
| FD and C Yellow No. 6 | 0.1% | w/w | — | |
| Aluminum Lake 5285 | | | | |
| Cab-O-Sil (M-5) | 0.5% | w/w | 0.5% | w/w |
| Stearic Acid, NF | 5.0% | w/w | 5.0% | w/w |
| (Powder) | | | | |
| THEORETICAL | 180 | mg | 150 | mg |
| TABLET WEIGHT | | | | |

The in vitro release rates of formulations 5 and 6, set forth in Table 3, were assessed by the USP Basket Method described hereinabove. Each of the formulations contained a total of 30 mg of oxycodone hydrochloride. The release rate of the oxycodone from each of the preparations is set forth below in Table 4.

TABLE 4

| TIME | FORMULA | FORMULA |
|---|---|---|
| (HOURS) | 5 (% LA) | 6 (% LA) |
| 0 | 0 | 0 |
| 1 | 20 | 24.3 |
| 2 | 28 | 35.8 |
| 4 | 41 | 55.1 |
| 6 | 50 | 67.3 |
| 8 | 58 | 76.3 |
| 10 | 64 | 82.5 |
| 12 | 70 | N/A |

Using methods similar to those described herein above, formulations according to the present invention are also made for tablets having 30 mg, 60 mg and 120 mg of Oxycodone Hydrochloride. Such formulation are set forth in Table 5.

TABLE 5

| | | 30 mg | 60 mg | 120 mg |
|---|---|---|---|---|
| | | strength | strength | strength |
| | | % w/w | % w/w | % w/w |
| Ingredient | Function | Lot G1051-01 | Lot G1051-07 | Lot G1051-12 |
| Oxycodone Hydrochloride, USP | Active Ingredient | 16.7 | 20 | 29.9 |
| Lactose, NF (Fast Flo) | Diluent | 12.8 | 12 | — |
| Methocel K100M (Premium) CR | SR Matrix Former | 55 | 55 | 44.8 |
| (Hydroxypropyl Methylcellulose, USP) | | | | |
| Sodium Polystyrene Sulfonate, | SR Matrix Aid | 10 | 7.5 | 19.9 |
| USP 27 μm Fine Particle Size | | | | |
| Cab-O-Sil (M-5) | Glidant | 0.5 | 0.5 | 0.5 |
| Stearic Acid, NF (Powder) | Lubricant | 5 | 5 | 5 |
| Alcohol SDA 23A | Granulating solution | * | — | * |
| Water, Purified, USP | Granulating solution | * | — | * |

| | | | |
|---|---|---|---|
| THEORETICAL TABLET WEIGHT | 180 mg | 300 mg | 402 mg |

* Removed during drying

Manufacturing Process
A. Tablets with 30 mg Oxycodone Hydrochloride:

Oxycodone hydrochloride, USP, Lactose, NF (Fast Flo), and Sodium Polystyrene Sulfonate, USP 27 μm Fine Particle Size and Methocel K100M (Premium) CR (Hydroxypropyl Methylcellulose, USP) are passed through a #20 mesh screen for delumping and are mixed for 20 minutes. The Water, Purified, USP and Alcohol, SDA 23 A are added to a tank and mixed. With the mixer running, the granulating fluid is added to the powder blend and the mixture is granulated. The wet mass is passed through a No. 10 mesh screen, placed back into the mixer and dried at 49° C. The dried granulation is passed through a mill. The Cab-O-Sil (M-5) is passed through a #20 mesh screen for delumping, then added to the drug powder blend and mixed for 5 minutes. The Stearic Acid, NF (Powder) is passed through a #40 mesh screen and then added to the batch. The batch is mixed for 5 minutes. Tablets are compressed using 5/16 inch tooling at a weight of 180 mg.

B. Tablets with 60 mg Oxycodone Hydrochloride:

Oxycodone hydrochloride, USP, Lactose, NF (Fast Flo), and Sodium Polystyrene Sulfonate, USP 27 μm Fine Particle Size are passed through a #20 mesh screen for delumping and are mixed for 10 minutes in a Bin. The Methocel K100M (Premium) CR (Hydroxypropyl Methylcellulose, USP) and Cab-O-Sil (M-5) are passed through a #20 mesh screen for delumping then added to the drug powder blend and mixed for 20 minutes in a Bin. The Stearic Acid, NF (Powder) is passed through a #40 mesh screen and then added to the batch. The batch is mixed for 5 minutes. Tablets are compressed using 11/32 inch tooling at a weight of 300 mg.

C. Tablets with 120 mg Oxycodone Hydrochloride:

Oxycodone Hydrochloride, USP, Sodium Polystyrene Sulfonate, USP 27 μm Fine Particle Size and Methocel K100M (Premium) CR (Hydroxypropyl Methylcellulose, USP) are passed through a #20 mesh screen for delumping and are mixed for 20 minutes. The Water, Purified, USP and Alcohol, SDA 23A are added to a tank and mixed. With the mixer running, the granulating fluid is added to the powder blend and the mixture is granulated. The wet mass is passed through a No. 10 mesh screen, placed back into the mixer and dried at 49° C. The dried granulation is passed through a mill. The Cab-O-Sil (M-5) is passed through a #20 mesh screen for delumping, then added to the drug powder blend and mixed for 5 minutes. The Stearic Acid, NF (Powder) is passed through a #40 mesh screen and then added to the batch. The batch is mixed for 5 minutes. Tablets are compressed using ⅜ inch tooling at a weight of 402 mg.

Dissolution Profiles

USP Basket Method at 100 rpm in 900 mL aqueous buffer (pH 1.2 for the first hour and 7.5 for hours 2-24) at 37° C. was used. The results are provided in Table 6.

TABLE 6

| | % Dissolved | | |
|---|---|---|---|
| Time (hrs) | 30 mg | 60 mg | 120 mg |
| 0 | 0 | 0 | 0 |
| 1 | 19 | 19 | 14 |
| 2 | 27 | 28 | 18 |
| 6 | 47 | 51 | 31 |
| 12 | 65 | 70 | 45 |
| 24 | 86 | 88 | 62 |

Example formulations of Oxycodone Hydrochloride Sustained Release Tablets (10 mg of active) were prepared using various particle sizes of Amberlite IRP 69. The specific formulations are set forth in Table 7. The function of each ingredient is also described.

TABLE 7

| | | Wa-P2-26 | Wa-P2-39 |
|---|---|---|---|
| Ingredient | Function | % w/w | % w/w |
| Oxycodone Hydrochloride, | Active | 6.7 | 6.7 |
| USP | Ingredient | | |
| Lactose, NF (Fast Flo) | Diluent | 27.8 | 27.8 |
| Methocel K100M (Premium) | SR Matrix | 55 | 55 |
| CR (Hydroxypropyl | Former | | |
| Methylcellulose, USP) | | | |
| Amberlite IRP 69 | SR Matrix | 5 | — |
| (Sodium Polystyrene | Aid | | |
| Sulfonate, USP) | | | |
| Amberlite IRP 69 | SR Matrix | — | 0.5 |
| (Sodium Polystyrene | Aid | | |
| Sulfonate, USP) sieve | | | |
| fraction retained on | | | |
| 100 mesh screen | | | |
| Amberlite IRP 69 | SR Matrix | — | 4.5 |
| (Sodium Polystyrene | Aid | | |
| Sulfonate, USP) sieve | | | |
| fraction through 325 | | | |
| mesh screen | | | |
| Cab-O-Sil (M-5) | Glidant | 0.5 | 0.5 |
| Stearic Acid, NF | Lubricant | 5 | 5 |
| (Powder) | | | |

| | | |
|---|---|---|
| THEORETICAL TABLET WEIGHT | 150 mg | 150 mg |

Manufacturing Process

Oxycodone Hydrochloride, USP, Lactose, NF (Fast Flo), and Amberlite IRP 69 (Sodium Polystrene Sulfonate, USP) are passed through a #20 mesh screen for delumping and are mixed for 10 minutes. The Methocel K100M (Premium) CR (Hydroxypropyl Methycellulose, USP) and Cab-O-Sil (M-5) are passed through a #20 mesh screen for delumping, then added to the drug powder blend and mixed for 20 minutes. The Stearic Acid, NF (Powder) is passed through a #40 mesh screen and then added to the batch. The batch is mixed for 5 minutes. Tablets are compressed using 9/32 inch tooling at a weight of 150 mg.

Dissolution Profiles (in Intestinal Solution)

USP Basket Method at 100 rpm in 900 mL aqueous buffer (pH 7.5) at 37° C. was used. The results are provided in Table 8.

TABLE 8
Oxycodone SR Tablets 10 mg % dissolved

| Time (hrs) | Lot Wa-P2-26 | Lot Wa-P2-39 |
|---|---|---|
| 0 | 0 | 0 |
| 1 | 25 | 23 |
| 2 | 37 | 33 |
| 4 | 55 | 49 |
| 6 | 68 | 61 |
| 8 | 79 | 72 |
| 10 | 87 | 83 |
| 12 | 94 | 92 |

Particle Size Data for various grades and sieve fractions of Amberlite IRP 69 (Sodium Polystyrene Sulfonate, USP) are set forth in Table 9.

TABLE 9

| | Mean | Particle size | Particle size |
|---|---|---|---|
| | particle | (US Standard | range |
| Grade | size(μm) | mesh) | (microns) |
| Amberlite IRP 69M | 27 | NLT 90% | <2 to 97 |
| (Sodium Polystyrene | | through | |
| Sulfonate, USP) | | 325 mesh | |
| Fine Particle Size | | | |
| Amberlite IRP 69 | 57 | 100-400 | <10 to 228 |
| (Sodium Polystyrene | | | |
| Sulfonate, USP) | | | |
| Amberlite IRP 27 μm | 27 | NLT 90% | <2 to 81 |
| Fine Particle Size | | through | |
| | | 325 mesh | |
| Amberlite IRP 69 | 23* | 100% | <5 to 53* |
| (Sodium Polystyrene | | through | |
| Sulfonate, USP) | | 325 mesh | |
| sieved through 325 | | | |
| mesh | | | |

NLT = Not Less Than
*Electrozone Fine Particle Size Analysis (Coulter principle) Particle Technology Labs, Ltd.

While the invention has been described with respect to preferred embodiments, those skilled in the art will readily appreciate that various changes and/or modifications can be made to the invention without departing from the spirit or scope of the invention as defined by the appended claims.

1-43. (canceled)

44. A solid, oral, controlled release dosage form consisting of a therapeutically effective amount of oxycodone or oxycodone hydrochloride between about 30 and 65% by weight of a matrix-forming polymer selected from the group consisting of hydroxypropyl cellulose, hydroxypropylmethyl cellulose and hydroxyethyl cellulose and between about 1 and 20% by weight of a cationic exchange resin having a mean particle size of less than about 50 μm and a particle size distribution such that not less than 90% of the particles pass through a 325 mesh sieve, U.S. Standard Sieve Size, wherein the oxycodone or oxycodone hydrochloride, the polymer and the cationic exchange resin are admixed with one another in dry form and then compressed

45. The dosage form of claim 44 wherein the cationic exchange resin comprises a sulfonated polymer.

46. The dosage form of claim 44 wherein the cationic exchange resin comprises a copolymer of divinyl-benzene and styrene.

47. The dosage form of claim 44 wherein the cationic exchange resin comprises a copolymer of divinylbenzene and methacrylic acid.

48. The dosage form of claim 44 wherein the cationic exchange resin comprises phenolic-based polyamine condensates.
